(19) United States
(12) Patent Application Publication
(10) Pub. No.: US 2025/0090297 A1
(43) Pub. Date: Mar. 20, 2025

(54) Vascular Implant

(71) Applicant: BARD PERIPHERAL VASCULAR, INC., Franklin Lakes, NJ (US)

(72) Inventors: Matt Casiraro, Tempe, AZ (US); Alexander Lastovich, Gilbert, AZ (US)

(73) Assignee: BARD PERIPHERAL VASCULAR, INC., Franklin Lakes, NJ (US)

(21) Appl. No.: 18/724,278

(22) PCT Filed: Dec. 30, 2021
(86) PCT No.: PCT/US2021/073179

§ 371 Date: Jun. 26, 2024

(51) Int. Cl. A61F 2/01; B33Y 80/00

(52) U.S. Cl. CPC A61F 2/012; B33Y 80/00; A61F 2220/0016; A61F 2220/0025; A61F 2230/0069; A61F 2240/001

(57) ABSTRACT

Avascular implant body having opposed filter ends and a central longitudinal axis. The body having a generally tubular, segmented side wall surrounding a central open-ended bore, the side wall includes a plurality of spaced apart wall sections. There is a filtering element in the bore in between the filter ends. Each tubular body end has a barbed vessel engagement members with opposed end portions, each end portion having one or more barbs. Each vessel engagement member has a concave portion in between the end portions.

### CROSS-REFERENCE TO RELATED APPLICATIONS

Not applicable

### STATEMENT REGARDING FEDERALLY SPONSORED RESEARCH OR DEVELOPMENT

Not applicable

### REFERENCE TO A “MICROFICHE APPENDIX”

Not applicable

### BACKGROUND OF THE INVENTION

### 1. Field of the Invention

The present invention relates to bioresorbable vascular implants such as filters (e.g., vena cava filters), occlusion devices, and convertible stents. In one preferred embodiment of the present invention, an improved vascular implant can bioresorb into a patient's vascular system (e.g., inferior vena cava, artery, vein or smaller vessel). For the inferior vena cava or iliofemoral vein, the implant can resorb after transient risk of pulmonary embolism (PE) has subsided. In one preferred embodiment of the present invention, the entire implant structure could be made of bioresorbable material so that no implant or implant remnant/element would ultimately be left behind as the entire implant would preferably resorb into the vascular tissue. In one preferred embodiment of the present invention, the implant is preferably specifically configured and 3d printable.

In one preferred embodiment of the present invention, a tubular implant body preferably includes multiple circumferentially spaced apart peripheral wall sections or panels, a centrally positioned ring, multiple appendages that connect the ring to the peripheral wall sections or panels, and opposed anchors, each positioned on a different one of the wall sections or panels (e.g., 180 degrees apart).

### 2. General Background of the Invention

Vascular implants include various devices that are placed at a selected locale in a patient's blood vessel. One example is a vena cava filter. Other examples include occlusion devices, stents, or convertible stents. Various patents have issued for vascular implants. Patents have also issued that relate in general to 3d printing of implants. Examples are listed in the following Table 1. Each patent or publication listed in Table 1 is hereby incorporated herein by reference.

TABLE 1

| PATENT OR | | ISSUE DATE |
|---|---|---|
| PUBLICATION NO. | TITLE | (DD/MM/YYYY) |
| 8,092,484 | EMBOLUS BLOOD CLOT FILTER | 10 Jan. 2012 |
| | WITH POST DELIVERY ACTUATION | |
| 8,092,485 | RECOVERABLE INFERIOR VENA | 10 Jan. 2012 |
| | CAVA FILTER | |
| 8,317,818 | REMOVABLE BLOOD CLOT FILTER | 27 Nov. 2012 |
| | WITH EDGE FOR CUTTING THROUGH | |
| | THE ENDOTHELIUM | |
| 8,420,113 | BIODEGRADABLE MEDICAL | 16 Apr. 2013 |
| | DEVICES WITH ENHANCED | |
| | MECHANICAL STRENGTH AND | |
| | PHARMACOLOGICAL FUNCTIONS | |
| 8,518,072 | JUGULAR FEMORAL VENA CAVA | 27 Aug. 2013 |
| | FILTER SYSTEM | |
| 8,562,638 | EMBOLUS BLOOD CLOT FILTER | 22 Oct. 2013 |
| | WITH FLOATING FILTER BASKET | |
| 8,734,479 | EMBOLUS BLOOD CLOT FILTER | 27 May 2014 |
| | DELIVERY SYSTEM | |
| 8,777,975 | EMBOLUS BLOOD CLOT FILTER | 15 Jul. 2014 |
| | WITH BIO-RESORBABLE COATED | |
| | FILTER MEMBERS | |
| 8,795,351 | MIGRATION RESISTANT EMBOLIC | 5 Aug. 2014 |
| | FILTER | |
| 8,870,943 | STENT STRUCTURE FOR | 28 Oct. 2014 |
| | IMPLANTATBLE MEDICAL DEVICE | |
| 8,992,562 | FILTER DELIVERY SYSTEM | 31 Mar. 2015 |
| 9,220,588 | SYSTEMS, METHODS AND DEVICE | 29 Dec. 2015 |
| | FOR EMBOLIC PROTECTION | |
| 9,393,095 | JUGULAR FEMORAL VENA CAVA | 19 Jul. 2016 |
| | FILTER SYSTEM | |
| 9,421,081 | EMBOLUS BLOOD CLOT FILTER | 23 Aug. 2016 |
| | DELIVERY SYSTEM | |
| 9,445,895 | INTRACARDIAC CAGE AND METHOD | 20 Sep. 2016 |
| | OF DELIVERING SAME | |
| 9,456,888 | REVERSIBLE VASCULAR FILTER | 4 Oct. 2016 |
| | DEVICES AND METHODS FOR USING | |
| | SAME | |
| 9,468,513 | EMBOLUS BLOOD CLOT FILTER | 18 Oct. 2016 |
| | WITH BIO-RESORBABLE COATED | |
| | FILTER MEMBERS | |
| 9,561,094 | DEVICES AND METHODS FOR | 7 Feb. 2017 |
| | TREATING VENOUS DISEASES | |
| 9,597,435 | MEDICAL DEVICES HAVING A | 21 Mar. 2017 |
| | BIORESORBABLE COATING LAYER | |
| | WITH A PRE-DETERMINED PATTERN | |
| | FOR FRAGMENTATION | |
| 9,693,851 | FILTER DELIVERY SYSTEM | 4 Jul. 2017 |
| 9,730,781 | EMBOLUS BLOOD CLOT FILTER | 15 Aug. 2017 |
| | REMOVAL SYSTEM AND METHOD | |
| 9,949,816 | IVC FILTER RETRIEVAL SYSTEMS | 24 Apr. 2018 |
| | WITH MULTIPLE CAPTURE MODES | |
| 9,980,804 | VENA CAVA FILTER WITH FILAMENT | 29 May 2018 |
| 10,105,206 | INFERIOR VENA CAVA FILTER WITH | 23 Oct. 2018 |
| | STABILITY FEATURES | |
| 10,188,496 | VENA CAVA FILTER FORMED FROM | 29 Jan. 2019 |
| | A SHEET | |
| 10,188,498 | EMBOLUS BLOOD CLOT FILTER | 29 Jan. 2019 |
| | DELIVERY SYSTEM | |
| 10,226,322 | JUGULAR FEMORAL VENA CAVA | 12 Mar. 2019 |
| | FILTER SYSTEM | |
| 10,258,454 | VISUAL STABILIZER ON ANCHOR | 16 Apr. 2019 |
| | LEGS OF VENA CAVA FILTER | |
| 10,279,078 | CROSSLINKABLE 3D PRINTED | 7 May 2019 |
| | BIOMATERIAL-BASED IMPLANTS | |
| | AND METHODS OF MANUFACTURE | |
| | THEREOF | |
| 10,299,906 | EMBOLUS BLOOD CLOT FILTER | 28 May 2019 |
| | UTILIZABLE WITH SINGLE DELIVERY | |
| | SYSTEM OR A SINGLE RETRIEVAL | |
| | SYSTEM IN ONE OF A FEMORAL OR | |
| | JUGULAR ACCESS | |
| 10,342,654 | IVC FILTER WITH TRANSLATING | 9 Jul. 2019 |
| | HOOKS | |
| 10,368,972 | EMBOLUS BLOOD CLOT FILTER | 6 Aug. 2019 |
| | WITH BIO-RESORBABLE COATED | |
| | FILTER MEMBERS | |
| 10,390,925 | MIGRATION RESISTANT EMBOLIC | 27 Aug. 2019 |
| | FILTER | |
| 10,441,689 | METHODS AND DEVICES FOR THREE- | 15 Oct. 2019 |
| | DIMENSIONAL PRINTING OR | |
| | ADDITIVE MANUFACTURING OF | |
| | BIOACTIVE MEDICAL DEVICES | |
| 10,470,865 | VASCULAR FILTER DEVICE | 12 Nov. 2019 |
| 10,492,898 | EMBOLUS BLOOD CLOT FILTER AND | 3 Dec. 2019 |
| | DELIVERY SYSTEM | |
| 10,512,531 | FILTER DELIVERY SYSTEM | 24 Dec. 2019 |
| 10,531,942 | ABSORBABLE VASCULAR FILTER | 14 Jan. 2020 |
| 10,579,755 | METHOD FOR 3-D PRINTING A | 3 Mar. 2020 |
| | CUSTOM BONE GRAFT | |
| 10,624,731 | VASCULAR FILTER SYSTEM | 21 Apr. 2020 |
| 10,729,527 | REMOVABLE EMBOLUS BLOOD | 4 Aug. 2020 |
| | CLOT FILTER | |
| 10,813,738 | TUBULAR FILTER | 27 Oct. 2020 |
| 10,842,608 | VENA CAVA FILTER WITH FILAMENT | 24 Nov. 2020 |
| 2007/0064731 | TRANSMISSION APPARATUS WITH | 22 Mar. 2007 |
| | FUNCTION OF MULTI-STEP | |
| | BANDWIDTH ASSIGNMENT TO | |
| | OTHER COMMUNICATION | |
| | APPARATUSES | |
| 2010/0074934 | MEDICAL IMPLANTS WITH A | 25 Mar. 2010 |
| | COMBINATION OF COMPOUNDS | |
| 2016/0166371 | ENDOLUMINAL FILTER DESIGN | 16 Jun. 2016 |
| | VARIATIONS | |
| 2016/0175085 | ENHANCED FLUOROGENIC | 23 Jun. 2016 |
| | ENDOLUMINAL FILTER STRUCTURE | |
| 2017/0105830 | BIODEGRADABLE VASCULAR | 20 Apr. 2017 |
| | FILTER | |
| 2017/0218228 | THREE DIMENSIONAL PRINTING OF | 3 Aug. 2017 |
| | BIO-INK COMPOSITIONS | |
| 2017/0249440 | 3D PRINTING SURGICAL REPAIR | 31 Aug. 2017 |
| | SYSTEMS | |
| 2017/0340429 | VASCULAR FILTER SYSTEM | 30 Nov. 2017 |
| 2018/0168811 | NOVEL BIODEGRADABLE AND NON- | 21 Jun. 2018 |
| | BIODEGRADABLE 3D PRINTED | |
| | IMPLANTS AS A DRUG DELIVERY | |
| | SYSTEM | |
| 2018/0296343 | 3-D PRINTING OF POROUS IMPLANTS | 18 Oct. 2018 |
| 2018/0303616 | 3-D PRINTING OF BONE GRAFTS | 25 Oct. 2018 |
| 2018/0311028 | VENA CAVA FILTER WITH FILAMENT | 1 Nov. 2018 |
| 2019/0110880 | MEDICAL DEVICES AND ANCHORS | 18 Apr. 2020 |
| | THREFOR | |
| 2020/0001540 | ADDITIVE MANUFACTURING ON | 2 Jan. 2020 |
| | UNCONSTRAINED FREEFORM | |
| | SURFACES | |
| 2020/0197150 | VASCULAR FILTER SYSTEM | 25 Jun. 2020 |
| WO2007064731 | HELICAL VENA CAVA FILTER | 7 Jun. 2007 |
| WO2011079287 | REVERSIBLE VASCULAR FILTER | 30 Jun. 2011 |
| | DEVICES AND METHODS FOR USING | |
| | SAME | |
| WO2016154148 | ARTIFICIAL TYMPANIC MEMBRANE | 29 Sep. 2016 |
| | DEVICES AND USES | |
| WO2018117907 | SHAPE MEMORY POLYMER | 28 Jun. 2018 |
| | COMPOSITE FOR 3D PRINTING OF | |
| | MEDICAL ITEMS | |
| WO2018218085 | THREE-DIMENSIONAL PRINTED | 29 Nov. 2018 |
| | ORGANS, DEVICES, AND MATRICES | |
| WO2019178086 | ELECTROHYDRODYNAMIC | 19 Sep. 2019 |
| | BIOPRINTER SYSTEM AND METHOD | |
| WO2020123945 | FABRIC MATERIAL FOR MEDICAL | 18 Jun. 2020 |
| | DEVICES | |
| EP2363156 | METHOD OF FABRICATING | 7 Sep. 2011 |
| | BIODEGRADABLE MEDICAL | |
| | DEVICES WITH ENHANCED | |
| | MECHANICAL STRENGTH AND | |
| | PHARMACOLOGICAL FUNCTIONS | |

### BRIEF SUMMARY OF THE INVENTION

The present invention provides a vascular implant preferably having opposed filter ends and a central longitudinal axis.

In one or more embodiments, the body preferably has a generally tubular, segmented side wall surrounding a central open ended bore, the side wall preferably including a plurality of spaced apart wall sections.

In one or more embodiments, a filtering element in the bore is preferably in between the filter ends.

In one or more embodiments, the tubular body end preferably has barbed vessel engagement members with opposed end portions, each end portion preferably having one or more barbs.

In one or more embodiments, each vessel engagement member preferably has a concave portion in between the end portions.

In one or more embodiments, the filter body, filtering element and barbed engagement members are preferably of a 3D printed polymeric construction.

In one or more embodiments, each barbed vessel engagement member preferably connects to a wall panel.

In one or more embodiments, the tubular sidewall is preferably generally cylindrically shaped.

In one or more embodiments, the vessel engagement member is preferably generally U-shaped.

In one or more embodiments, the filtering element preferably includes multiple circumferentially spaced apart, radially extending arms.

In one or more embodiments, the filtering element preferably includes a hub.

In one or more embodiments, the filtering element preferably includes a ring.

In one or more embodiments, the filtering elements preferably include multiple circumferentially spaced apart arms, each arm preferably connecting to a wall panel and to a hub.

In one or more embodiments, the tubular implant body preferably has opposed filter ends and a central longitudinal axis.

In one or more embodiments, multiple filtering elements in the bore preferably include an annular member and multiple arms, each extending from the annular member to a wall section.

In one or more embodiments, the filter body, filtering elements and barbed engagement members are preferably of a 3D printed polymeric construction.

In one or more embodiments, the annular member is preferably a ring.

In one or more embodiments, the annular member is preferably a hub.

In one or more embodiments, the annular member has a peripheral portion and each arm preferably attaches to the peripheral portion.

In one or more embodiments, the body can be too soft to be machined.

In one or more embodiments, the body can be made of a material with a durometer reading between about 10 Shore A and 75 Shore D.

In one or more embodiments, the body can be made of a material with a durometer reading between about 10 Shore A and 40 Shore A.

In one or more embodiments, the body can be made of a material with a durometer reading between about 65 Shore A and 75 Shore D.

In one or more embodiments, the body preferably has a generally tubular, segmented side wall surrounding a central open ended bore, the side wall preferably includes a plurality of spaced apart wall sections.

In one or more embodiments, the filtering element in the bore is preferably in between the filter ends,

In one or more embodiments, the tubular body end preferably has barbed vessel engagement members with opposed end portions, each end portion preferably has one or more barbs.

In one or more embodiments, the vessel engagement member preferably has a central portion attached to a wall section and wherein the end portions are preferably spaced away from any of the wall section.

In one or more embodiments, each vessel engagement member preferably includes a curved panel.

In one or more embodiments, the curved panel preferably has a convex portion attached to a wall panel and a concave portion.

In one or more embodiments, the barbs are preferably extended outwardly of the concave portion.

In one or more embodiments, each curved panel preferably has a pair of upper barbs and a pair of lower barbs.

In one or more embodiments, each filtering member is connected to a central hub with a ball and socket connection.

In one or more embodiments, each filtering member is connected to each wall section with a ball and socket connection.

In one or more embodiments, each filtering member is connected to each wall section and a central hub with ball and socket connections.

In one or more embodiments, each filtering member passes through a bore which can be conically or frusto-conically shaped.

### BRIEF DESCRIPTION OF THE SEVERAL VIEWS OF THE DRAWINGS

For a further understanding of the nature, objects, and advantages of the present invention, reference should be had to the following detailed description, read in conjunction with the following drawings, wherein like reference numerals denote like elements and wherein:

FIG. 1 is a side or elevation view of a preferred embodiment of the apparatus of the present invention;

FIG. 2 is a top or plan view of a preferred embodiment of the apparatus of the present invention;

FIG. 3 is a perspective view of a preferred embodiment of the apparatus of the present invention;

FIG. 4 is a fragmentary side view of a preferred embodiment of the apparatus of the present invention;

FIG. 5 is a fragmentary side view of a preferred embodiment of the apparatus of the present invention;

FIG. 6 is a fragmentary top view of a preferred embodiment of the apparatus of the present invention;

FIG. 7 is a perspective view of a second preferred embodiment of the apparatus of the present invention;

FIG. 8 is a top or plan view of a second preferred embodiment of the apparatus of the present invention;

FIG. 9 is a close-up, partial perspective view of a preferred embodiment of the apparatus of the present invention;

FIG. 10 is a perspective view of a third preferred embodiment of the apparatus of the present invention; and

FIG. 11 is a top or plan view of a third preferred embodiment of the apparatus of the present invention.

### DETAILED DESCRIPTION OF THE INVENTION

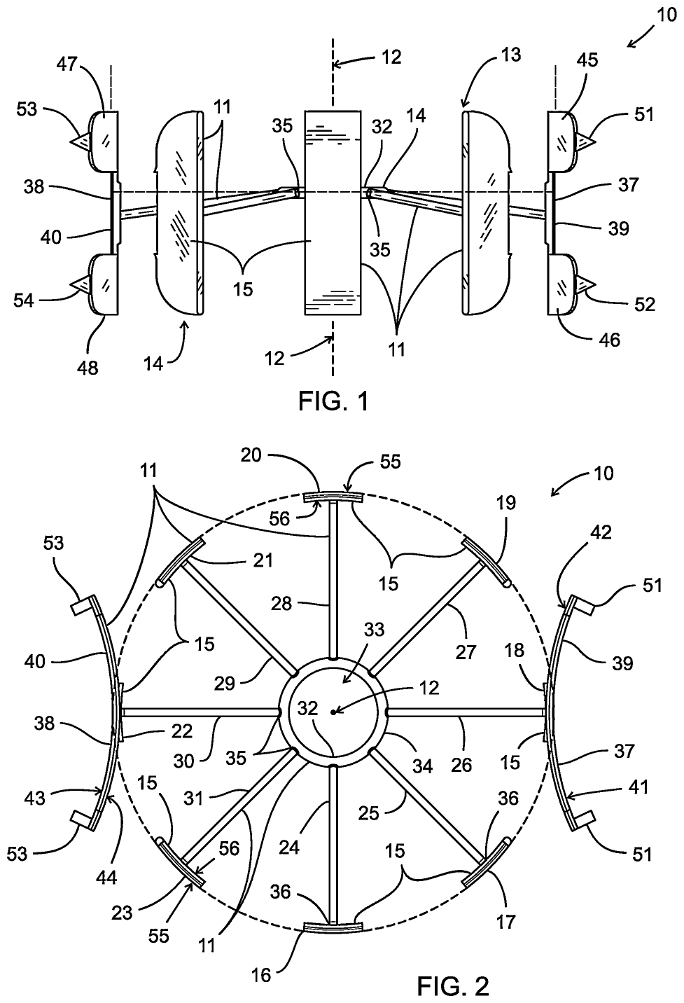

FIGS. 1-6 show a preferred embodiment of the apparatus of the present invention, designated generally by the numeral 10. The vascular implant 10 can e.g. be implemented as a vascular filter or as an occlusion device. Vascular implant 10 has an implant body 11 that can be bioresorbable, such as of a bioresorbable polymeric material. For inferior vena cava pulmonary embolism protection, the implant 10 can have a diameter of between about 15-30 mm. For iliofemoral pulmonary embolism protection, the implant 10 can have a diameter of between about 6-20 mm. For other smaller vessels for occlusion, the implant 10 in the form of an occlusion device can have a diameter of between about 2-8 mm. The implant body 10 has a central longitudinal axis 12, upper or proximal end 13 and lower or distal end 14 (see FIG. 1). Body 11 can be between about 5-50 mm long.

Implant body 11 has a segmented side wall 15. In this embodiment the side wall 15 is generally tubular. The segmented side wall comprises multiple circumferentially spaced apart wall panels or wall segments 16-23. Each wall panel or wall segment 16, 17, 18, 19, 20, 21, 22, 23 preferably connects with an arm or filtering member 24-31. Each segment 16-23 can have edges that are sharp, filleted or chamfered. Electropolishing can be used to provide the edges of each panel 16-23 with a rounded edge. The side wall 15 extends in circumferential direction around the central axis 12. The side wall 15 extends in axial direction from the upper or proximal end 13 to the lower or distal end 14 and defines and encloses, in circumferential direction around the central axis 12, a passage through which fluid, e.g. blood, transported through the vessel can flow, in this embodiment from the lower or distal end 14 to the upper or proximal end 13. The passage may also be described as a central, open-ended, bore, which is surrounded by the side wall 15.

As explained below in more detail, one or more filtering elements are present which block particles, e.g. blood clots, above a predetermined side from passing through the passage beyond the proximal end 13 while allowing the fluid to pass through the passage. In this embodiment, the filtering elements are located in the passage, and block particles that entered the passage from the distal end 14 from leaving the passage at the proximal end. In the shown embodiment of FIGS. 1-6, the filtering elements are arms or filtering members 24-31 which in circumferential direction are spaced apart. The arms or filtering members 24-31 may form an open cell filter, for instance. When the implant is placed in a vessel with the lower or distal end 14 upstream and the upper or proximal end 13 downstream, in the direction of fluid flow, blood will flow through the passage from the lower or distal end 14 to the upper or proximal end 13 and pass through the implant body 11. The fluid will flow through the voids between the arms or filtering members 24-31, as well as in this embodiment through the central opening 33 in hub, head, annular member or ring 32 preferably has, and blood clots that cannot pass through this space or through, if present, the opening are blocked from passing through the implant. Arms or filtering members 24-31 can be of a bioresorbable polymeric material. Arms or filtering members 24-31 can be about 5-50 mm long and each arm can have a width of about 0.1-10 mm.

In this embodiment, the wall panels or wall segments 16-23 are oriented parallel to the central longitudinal axis. The segments are in this embodiment oriented in “portrait’ mode Alternatively or additionally, some or all of the segments may be oriented in “landscape’ mode. The wall panels or wall segments 16-23 have an inwards facing surface which faces towards the central axis 12, and an outwards facing surface which faces away from the central axis. As shown, the wall panels or wall segments 16-23 may be oriented with their outwards facing surface extending parallel to the tangential direction, and the wall panels or wall segments 16-23 are in this embodiment located in a radial direction at the same distance from the central axis 12, thus forming a side wall shaped as a segmented cylinder, which in this example is an open cylinder. The cylinder extends from the bottom, formed by a short side of the segments to the top, formed by the opposite short side of the segments.

In the figures, one or more, in this embodiment all, arm or filtering member 24-31 can connect to a wall panel or wall segment 16-23. In the figures, one or more, in this embodiment all, arm or filtering member 24-31 can connect to hub, head, annular member or ring 32. Said differently, in this embodiment each arm or filtering member 24-31 connects a single wall segment 16-23 to hub, head, annular member or ring 32. Seen in axial direction, from the distal end 14 to the proximal end 13, the position at which the respect arm or filtering member 24-31 connects to the wall panel or wall segment 16-23 is below the position at which the arm or filtering member 24-31 connects to the hub, head, annular member or ring 32. Said differently, the arm or filtering members 24-31 extend from the hub outwards in radial direction and, opposite to the axial direction, from the hub in the direction of the distal end. In this embodiment, each of the arm or filtering members 24-31 is unbranched and not directly attached to the other arm or filtering members 24-31. Each of the arm or filtering members 24-31 can thus move independently from the other arm or filtering members 24-31. The risk of damage to the vessel by the implant can thereby be reduced.

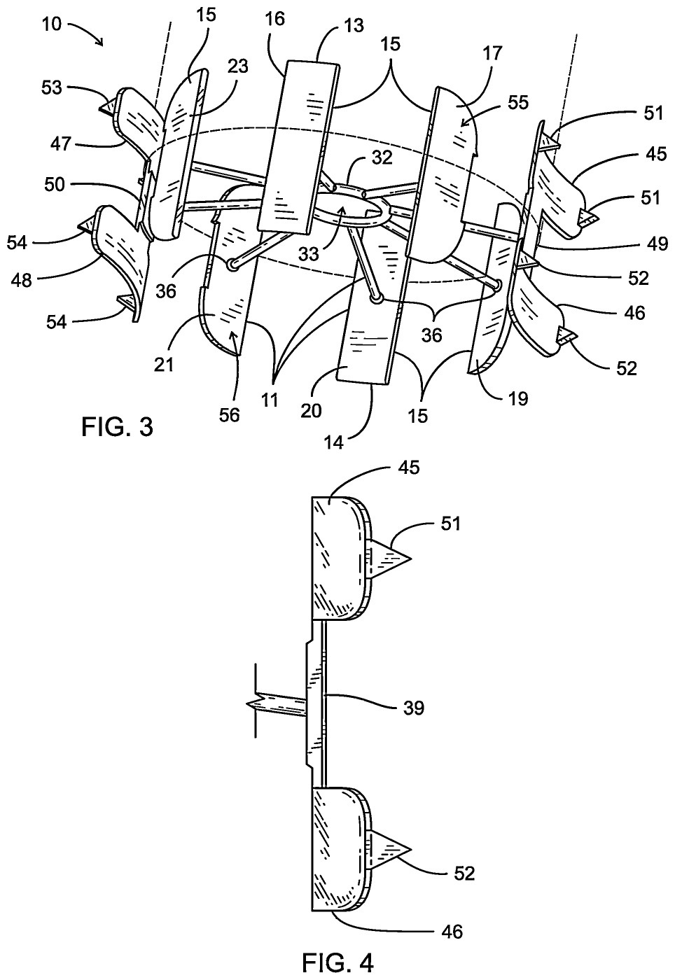

As can be seen in FIGS. 2 and 3, hub, head, annular member or ring 32 is located in the perimeter defined by the side wall 15, and lies, seen in radial direction, inwards, at a distance from the side wall 15. When the implant body 11 is positioned in a vessel, the hub, head, annular member or ring 32 lies in radial direction inwards, at a distance from the vessel wall. In this embodiment, the hub, head, annular member or ring 32 lies co-axially with the axis of the passage. In this embodiment, hub, head, annular member or ring 32 lies in the passage defined by the side wall 15, between the distal end 14 and the proximal end 13.

Hub, head, annular member or ring 32 preferably has central opening 33 and peripheral portion 34. Each arm or filtering member 24-31 preferably connects to hub, head, annular member or ring 32 at an attachment portion or joint 35 which can be placed at peripheral portion 34 as seen in FIGS. 1-2. Each arm or filtering member 24-31 connects to a wall panel or wall segment 16-23 with a panel attachment 36 (see FIGS. 2, 3). Each wall segment or wall panel 16-23 can have outer convex surface 55 and an inner concave surface 56. The panels 16-23 can reduce the risk of damage to the vessel and/or reduce perturbations in the flow profile. Surfaces 55,56 may be curved in the circumferential direction and not in the axial direction of the implant body 11. Preferably, but not necessarily, the curvature of the outer convex surface 55 and/or inner concave surface 56 in the circumferential direction is more or less the same as of a cylinder with a radius equal to the distance of the respective surface to the central axis 12. In FIG. 2, arm/filtering element 24 connects to wall panel or wall segment 16. Arm or filtering element 24 also connects to head, hub, ring, or annular member 32 peripheral portion 34. Similarly, arm 25 connects to panel 17, arm 26 connects to panel 18, arm 27 connects to panel 19, arm 28 connects to panel 20, arm 29 connects to panel 21, arm 30 connects to panel 22 and arm 31 connects to panel 23. Joints/attachments 35 can be integral with hub, head, annular member or ring 32 such as when body 11 is manufactured with 3d printing. Similarly, panel attachments 36 can be integral with arms/filtering members 24-31 as when body 11 is a one piece body made with a 3d printer. When fabricating body 11 using a 3d printer, the implant body 11 can be made of a bioresorbable polymer material. When of a bioresorbable material, the bioresorption starts as soon as the implant is exposed to the blood and like many of the standard polymers degrades over time via polymer breakdown and absorption of the byproducts. This bioresorption can be tuned as needed to ensure the implant maintains structural filtering integrity until transient risk of pulmonary embolism has subsided. Such a material is typically not hard enough to be machined. The implant body 11 can be made of a material having a durometer reading of between about 10 Shore A and 75 Shore D. In certain embodiments, the implant body 11 can be made of a material too soft to be machined (below approximately 20 Shore D).

Vessel engagement members or vessels anchors 37, 38 are provided on body 11, preferably spaced circumferentially apart as shown in FIGS. 1 and 2. In this embodiment, the vascular implant comprises a set of vessel engagement members or vessels anchors 37, 38, with the vessel engagement members or vessels anchors of the set being opposite to each other, at the lateral side of the central bore. Surprisingly, it has been found that two vessel engagement members or vessels anchors 37, 38 is already sufficient to reliably secure the vascular implant, although more vessel engagement members or vessels anchors may be present. When the implant is placed inside the vessel, the vessel engagement member or vessel anchor 37, 38 engage with the vessel to retain the implant in position and maintain the desired orientation of the implant. Each vessel engagement member or vessel anchor projects in radial direction relative to the side wall.

Each vessel engagement member or vessel anchor 37, 38 has opposed end portions, in this example at sides opposite in the axial direction. On each end portion one or more barbs 51-54 are provided. In this embodiment, each of the opposed end portions lies in axial direction at a respective one of the upper or proximal end 13 and lower or distal end 14 and forms a barbed vessel engagement member located at an end of the tubular body. The barbs 51-54 project radially outwards, and when the implant is placed inside the vessel point into the vessel wall. Because of the barbs, the risk of dislocation of the vascular implant can be reduced.

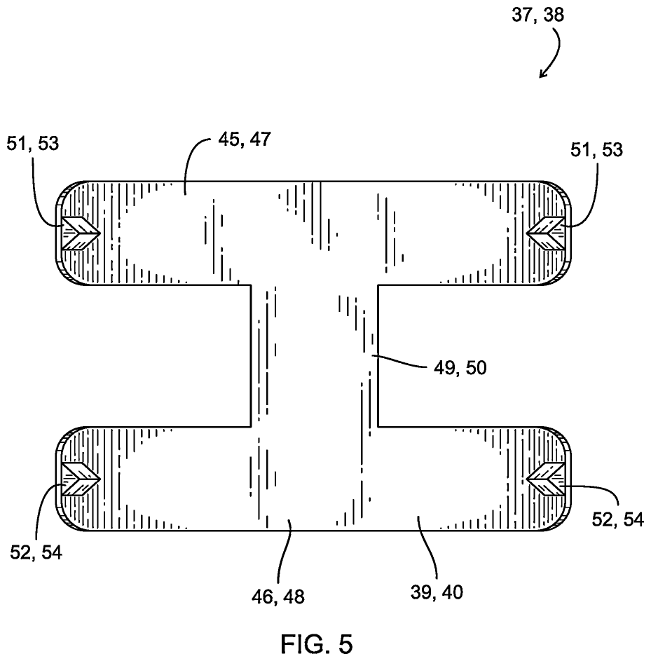

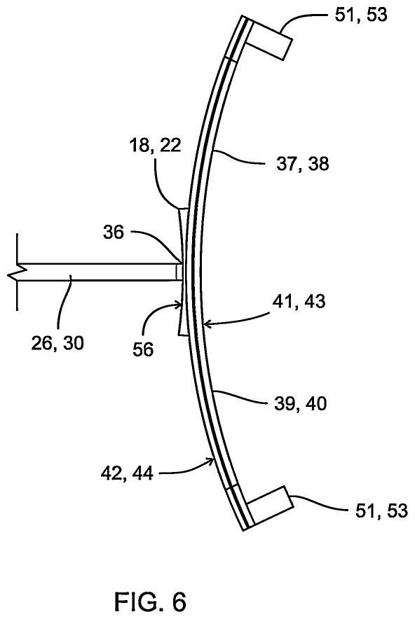

The vessel engagement member or vessel anchor 37, 38 may comprise a panel 39 or 40 fitted preferably with barbs, hooks, projections or points 51-54 (see FIG. 5). In the embodiments, the panels 39,40 are parallel to the side wall. Each panel 39, 40 has concave and convex surfaces. In this embodiment, the concave surface faces away from the body, and faces the wall of the vessel when the vascular implant is properly positioned in a vessel. The convex surface is opposite to the concave surface, and the convex surface faces the side wall and the central bore. Panel 39 has concave surface 41 and convex surface 42 (see FIGS. 2, 6). Panel 40 has concave surface 43 and convex surface 44. Each panel 39, 40 has upper and lower sections. Panel 39 has upper or proximal section 45 and lower or distal section 46. Panel 40 has upper or proximal section 47 and lower or distal section 48. Each panel 39, 40 may comprise a central or middle section with a reduced width of the panel 39,40. Said differently, each panel may be I-shaped, with an upper section and/or lower section where the width is larger than at the central or middle section, and the central or middle section being indented at both sides. Panel 39 has central or middle section 49. Panel 40 has central or middle section 50. Barbs, hooks, projections or points 51-54 are preferably provided on the concave surfaces 41, 43 of the panels 39, 40. Barbs 51 are preferably placed on upper section 45 of panel 39. Barbs 52 are preferably placed on lower section 46 of panel 39. Similarly, barbs 53 are upper barbs preferably placed on upper or proximal section 47 of panel 40, while barbs 54 are lower barbs preferably placed on concave surface 43 of panel 40 lower section 48. In this embodiment, the barbs are located on a respective extremity of the upper or lower section, which in the circumferential direction projects beyond the central or middle section. For example, each extremity, e.g. of the four extremities of an I-shape, may be provided with one or more barbs.

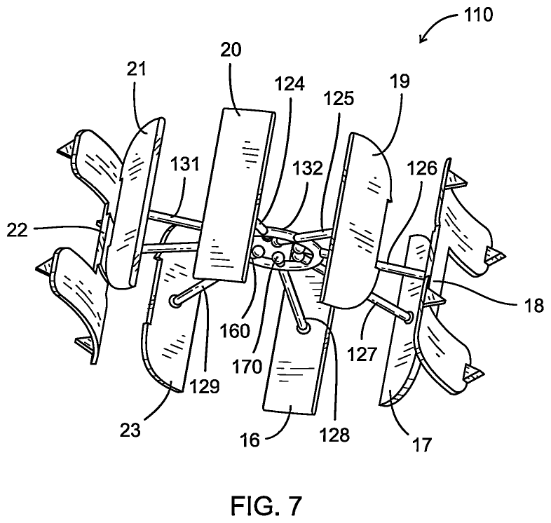

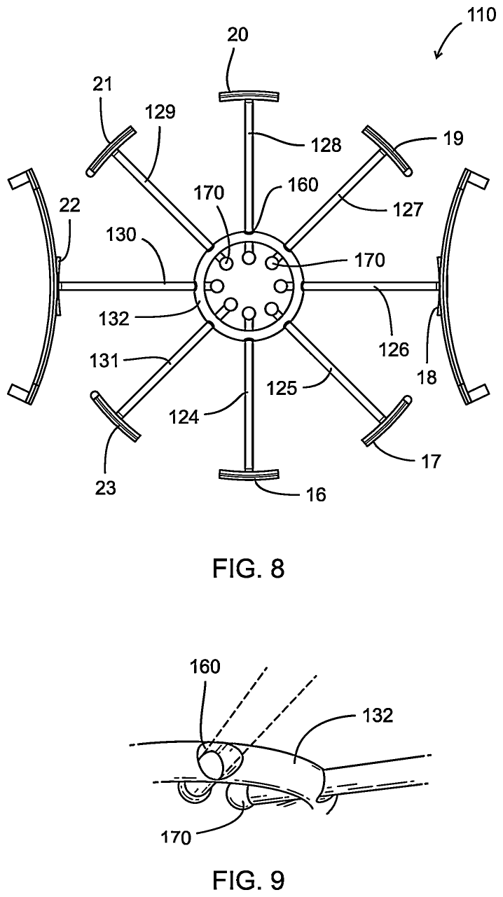

FIGS. 7-9 show a preferred embodiment of the apparatus of the present invention, designated generally by the numeral 110. Vascular implant 110 is similar to vascular implant 10 shown in FIGS. 1-6 and except as noted, contains the same parts. In vascular implant 110, each arm/filtering member 124-131 connects to each wall panel/wall segment 16-23 in the same manner as the embodiments shown in FIGS. 1-6. Each arm/filtering member 124-131 connects to annular member/ring/hub/head 132 by ball 170 and socket 160 connection. Each rounded end/ball 170 prevents each filtering member/arm from fully separating from annular member/ring/hub/head 132. The hole/socket/bore 160 through which each filtering member 124-131 passes can be conically or frusto-conically shaped, as depicted in FIG. 9, to allow for a change of angle of each filtering member 124-131 relative to hub 132, and accordingly, each wall section/panel 16-23. Hole/socket/bore 160 is open ended and thus allows for each section/panel 16-23 to be pulled inwardly toward the annular member/ring/hub/head 132, for example, when collapsing the vascular implant 110. The ball 170 and socket 160 connection or feature as shown in FIGS. 7-9 is preferably applicable to the vascular implant 110 on the higher end of the durometer range, for example when the material has a hardness that is within a durometer reading of between about 65 Shore A and 75 Shore D.

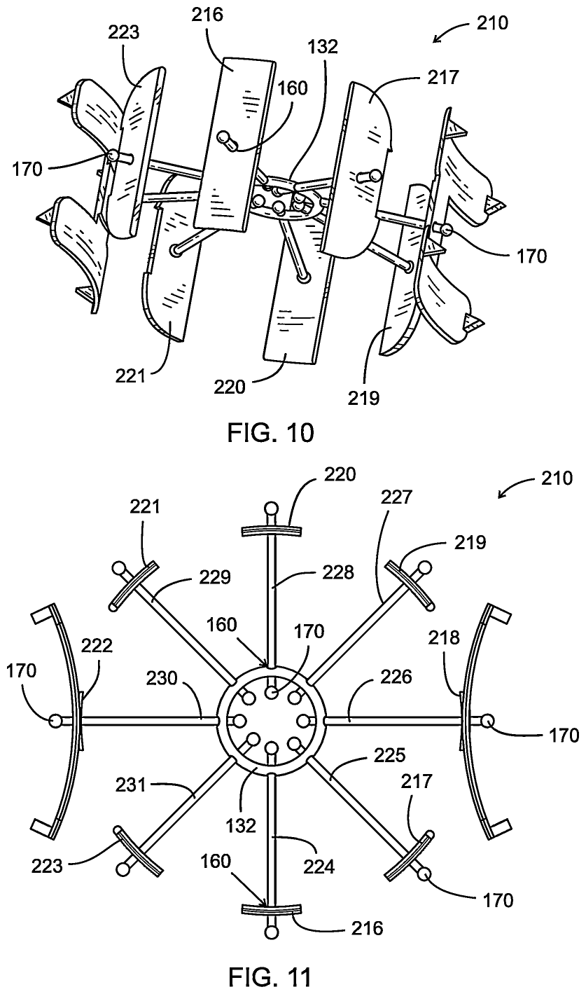

FIGS. 10-11 show another preferred embodiment of the apparatus of the present invention, designated generally by the numeral 210. Vascular implant 210 is similar to vascular implant 110 except as noted and depicted in the drawings. Each arm/filtering member 224-231 of vascular implant 210, unlike vascular implant 110, has a ball 170 and socket 160 connection to both the annular member/ring/hub/head 132 and each wall panel/wall segment 216-223 (vascular implant 110 as shown only has such a connection to the central annular member/ring/hub/head 132). Each of the plurality of wall panels/segments 216-223 of vascular implant 210 has a hole or socket or bore 160 for each filtering member/arm 224-231 to pass through. Each hole or socket or bore 160 on the outer wall is open ended and can be conically shaped in the same manner as annular member/ring/hub/head 132. The ball 170 and socket 160 feature on both ends, as shown in vascular implant 210 of FIGS. 10-11, allows for an angle change of the cross member/filtering member/arm 224-231 relative to both the annular member 132 as well as each corresponding wall segment/wall panel 216-223.

The vascular implant of the present invention can be of a material that has a hardness within a durometer reading of between about 10 Shore A and 75 Shore D. However, when the material is on the higher end of the durometer range, for example, when the material has a hardness that is within a durometer reading of between about 65 Shore A and 75 Shore D, the vascular implant preferably comprises the ball and socket connections of FIGS. 10-11. When there is an absence of ball and socket connections on either the central hub or outer walls, (e.g., FIGS. 1-8), the material preferably has a hardness within a durometer reading of between about 10 Shore A and 40 Shore A.

The implant 10, 110, 210 could be deployed with either a femoral or jugular approach. Such a deployment could employ a pusher or pusher apparatus/mechanism such as one specified in one or more of the patents listed in Table 1. An example is U.S. Pat. No. 8,518,072 naming Jonathan Miller as inventor and assigned to C.R. Bard, Inc. Implant 10 could also be a balloon-mounted implant that is then expanded with balloon dilation, as seen, for example, in balloon mounted stents.

The vascular implant can thus be characterized by comprising a tubular implant body with opposed filter ends and a central longitudinal axis and one or more filtering elements. The body comprises a generally tubular, segmented side wall surrounding a central open-ended bore. The side wall includes a plurality of spaced apart wall sections. The vascular implant further comprises barbed vessel engagement members with end portions comprising one or more barbs.

Without limitation, the implant may further be characterized by one or more of the following statements.
Statement 1: A vascular implant, comprising:
   a) a tubular implant body comprising opposed filter ends and a central longitudinal axis;
   b) the body comprising a generally tubular, segmented side wall surrounding a central open ended bore, the side wall including a plurality of spaced apart wall sections;
   c) a filtering element in the bore in between the filter ends;
   d) each the tubular body end comprising barbed vessel engagement members with opposed end portions, each end portion comprising one or more barbs; and
   e) wherein each vessel engagement member comprises a concave portion in between the end portions.
Statement 2: The vascular implant one or more of the preceding statements wherein the implant body, filtering element and barbed vessel engagement members are of a 3D printed polymeric construction.
Statement 3: The vascular implant one or more of the preceding statements wherein each the barbed vessel engagement member connects to a wall section.
Statement 4: The vascular implant one or more of the preceding statements wherein the tubular side wall is generally cylindrically shaped.
Statement 5: The vascular implant one or more of the preceding statements wherein the vessel engagement member is generally U-shaped.
Statement 6: The vascular implant one or more of the preceding statements wherein the filtering element includes multiple circumferentially spaced apart, radially extending arms.
Statement 7: The vascular implant one or more of the preceding statements wherein the filtering element includes a ring or hub.
Statement 8: The vascular implant one or more of the preceding statements wherein each the filtering element movably attaches to a wall section with a connection that enables the filtering element to pivot with respect to a wall section.
Statement 9: The vascular implant of statement 7 wherein the filtering elements include multiple circumferentially spaced apart arms, each arm connecting to a wall section and to the hub.
Statement 10: A vascular implant, comprising:
   a) a tubular implant body comprising opposed filter ends and a central longitudinal axis;
   b) the body comprising a generally tubular, segmented side wall surrounding a central open ended bore, the side wall including a plurality of spaced apart wall sections;
   c) multiple filtering elements in the bore including an annular member and multiple arms, each extending from the annular member to a the wall section;
   d) each the tubular body end comprising barbed vessel engagement members with opposed end portions, each end portion comprising one or more barbs; and
   e) wherein each vessel engagement member comprises a concave portion in between the end portions.
Statement 11: The vascular implant of statement 10 wherein the implant body, filtering elements and barbed engagement members are of a 3D printed polymeric construction.
Statement 12: The vascular implant of one or more of statements 10-11 wherein the annular member is a ring or hub.
Statement 13: The vascular implant of one or more of statements 10-12 wherein each arm connects to the annular member with a ball and socket connection.
Statement 14: The vascular implant of one or more of statements 10-13 wherein the annular member comprises a peripheral portion and each arm attaches to the peripheral portion.
Statement 15: A vascular implant, comprising:
   a) a tubular implant body comprising opposed filter ends and a central longitudinal axis;
   b) the body comprising a generally tubular, segmented side wall surrounding a central open ended bore, the side wall including a plurality of spaced apart wall sections;
   c) a filtering element in the bore in between the filter ends;
   d) each the tubular body end comprising barbed vessel engagement members with opposed end portions, each end portion comprising one or more barbs; and
   e) wherein each vessel engagement member comprises a central portion attached to a the wall section and wherein the end portions are spaced away from any of the wall section.
Statement 16: The vascular implant of statement 15 wherein the vessel engagement member is generally U-shaped.
Statement 17: The vascular implant of statement 15 or 16 wherein each vessel engagement member include a curved panel.
Statement 18: The vascular implant of statement 17 wherein the curved panel comprises a convex portion attached to a the wall panel and a concave portion.
Statement 19: The vascular implant of statement 18 wherein the barbs are extended outwardly of the concave portion.
Statement 20: The vascular implant of statement 17, 18 or 19 wherein each curved panel comprises a pair of upper barbs and a pair of lower barbs.

The following is a list of parts and materials suitable for use in the present invention.

PARTS LIST

| Part Number | Description |
|---|---|
| 10 | vascular implant |
| 11 | implant body |
| 12 | central longitudinal axis |
| 13 | upper end/proximal end |
| 14 | lower end/distal end |
| 15 | segmented side wall |
| 16 | wall panel/wall segment |
| 17 | wall panel/wall segment |
| 18 | wall panel/wall segment |
| 19 | wall panel/wall segment |
| 20 | wall panel/wall segment |
| 21 | wall panel/wall segment |
| 22 | wall panel/wall segment |
| 23 | wall panel/wall segment |
| 24 | arm/filtering member |
| 25 | arm/filtering member |
| 26 | arm/filtering member |
| 27 | arm/filtering member |
| 28 | arm/filtering member |
| 29 | arm/filtering member |
| 30 | arm/filtering member |
| 31 | arm/filtering member |
| 32 | hub/head/annular member/ring |
| 33 | central opening |
| 34 | peripheral portion |
| 35 | attachment/joint |
| 36 | panel attachment |
| 37 | vessel engagement member/vessel anchor |
| 38 | vessel engagement member/vessel anchor |
| 39 | panel |
| 40 | panel |
| 41 | concave surface |
| 42 | convex surface |
| 43 | concave surface |
| 44 | convex surface |
| 45 | upper section |
| 46 | lower section |
| 47 | upper section |
| 48 | lower section |
| 49 | central section |
| 50 | central section |
| 51 | upper barbs |
| 52 | lower barbs |
| 53 | upper barbs |
| 54 | lower barbs |
| 55 | convex surface |
| 56 | concave surface |
| 110 | vascular implant |
| 124 | arm/filtering member |
| 125 | arm/filtering member |
| 126 | arm/filtering member |
| 127 | arm/filtering member |
| 128 | arm/filtering member |
| 129 | arm/filtering member |
| 130 | arm/filtering member |
| 131 | arm/filtering member |
| 132 | hub/head/annular member/ring |
| 160 | hole/socket/conically shaped opening/bore |
| 170 | rounded end/ball |
| 210 | vascular implant |
| 216 | wall panel/wall segment |
| 217 | wall panel/wall segment |
| 218 | wall panel/wall segment |
| 219 | wall panel/wall segment |
| 220 | wall panel/wall segment |
| 221 | wall panel/wall segment |
| 222 | wall panel/wall segment |
| 223 | wall panel/wall segment |
| 224 | arm/filtering member |
| 225 | arm/filtering member |
| 226 | arm/filtering member |
| 227 | arm/filtering member |
| 228 | arm/filtering member |
| 229 | arm/filtering member |
| 230 | arm/filtering member |
| 231 | arm/filtering member |

All measurements disclosed herein are at standard temperature and pressure, at sea level on Earth, unless indicated otherwise. All materials used or intended to be used in a human being are biocompatible, unless indicated otherwise.

The foregoing embodiments are presented by way of example only; the scope of the present invention is to be limited only by the following claims.

1. A vascular implant, comprising:
a) a tubular implant body comprising opposed first and second filter ends and a central longitudinal axis;
b) said body comprising a generally tubular, segmented side wall surrounding a central open ended bore, said side wall including a plurality of spaced apart wall sections, each said wall section extending in between said first and second filter ends;
c) a filtering element in said bore in between said first and second filter ends;
d) each said tubular body comprising barbed vessel engagement members with opposed barbed vessel engagement end portions, each barbed vessel engagement end portion comprising one or more barbs; and
e) wherein each barbed vessel engagement member has a concave portion in between said barbed vessel engagement end portions.

2. The vascular implant of claim 1 wherein said implant body, filtering element and barbed vessel engagement members are of a 3D printed polymeric construction.

3. The vascular implant of claim 1 wherein each said barbed vessel engagement member connects to a said wall section.

4. The vascular implant of claim 1 wherein said tubular side wall is generally cylindrically shaped.

5. The vascular implant of claim 1 wherein said barbed vessel engagement member is generally U-shaped.

6. The vascular implant of claim 1 wherein said filtering element includes multiple circumferentially spaced apart, radially extending arms.

7. The vascular implant of claim 1 wherein said filtering element includes a ring or hub.

8. The vascular implant of claim 1 wherein each said filtering element movably attaches to a said wall section with a connection that enables the said filtering element to pivot with respect to a said wall section.

9. The vascular implant of claim 7 wherein the filtering elements include multiple circumferentially spaced apart arms, each arm connecting to a said wall section and to said hub.

10. A vascular implant, comprising:
a) a tubular implant body having opposed first and second filter ends and a central longitudinal axis;
b) said body having a generally tubular, segmented side wall surrounding a central open ended bore, said side wall including a plurality of spaced apart wall sections;
c) multiple filtering elements in said bore including an annular member and multiple arms, each extending from the annular member to a said wall section;
d) each said tubular body having one or more barbed vessel engagement members with opposed barbed vessel engagement end portions, each barbed vessel engagement end portion having one or more barbs; and
e) wherein each vessel engagement member has a concave portion in between said barbed vessel engagement end portions.

11. The vascular implant of claim 10 wherein said implant body, filtering elements and barbed engagement members are of a 3D printed polymeric construction.

12. The vascular implant of claim 10 wherein the annular member is a ring or hub.

13. The vascular implant of claim 10 wherein each arm connects to the annular member with a ball and socket connection.

14. The vascular implant of claim 10 wherein said annular member has a peripheral portion and each arm attaches to said peripheral portion.

15. A vascular implant, comprising:
a) a tubular implant body having opposed filter ends and a central longitudinal axis;
b) said body having a generally tubular, segmented side wall surrounding a central open ended bore, said side wall including a plurality of spaced apart wall sections;
c) a filtering element in said bore in between said filter ends;
d) each said tubular body end having barbed vessel engagement members with opposed barbed vessel engagement end portions, each barbed vessel engagement end portion having one or more barbs; and
e) wherein each vessel engagement member has a central portion attached to a said wall section and wherein said barbed vessel engagement end portions are spaced away from any of said wall sections.

16. The vascular implant of claim 15 wherein said vessel engagement member is generally U-shaped.

17. The vascular implant of claim 15 wherein each vessel engagement member include a curved panel.

18. The vascular implant of claim 17 wherein said curved panel has a convex portion attached to a said wall panel and a concave portion.

19. The vascular implant of claim 18 wherein said barbs are extended outwardly of said concave portion.

20. The vascular implant of claim 17 wherein each curved panel has a pair of upper barbs and a pair of lower barbs.

21. (canceled)
